(19) United States
(12) Patent Application Publication
(10) Pub. No.: US 2012/0302735 A1
(43) Pub. Date: Nov. 29, 2012

(54) PRODUCTION OF SOY PROTEIN PRODUCT

(76) Inventors: Brent E. Green, Warren (CA); Martin Schweizer, Winnipeg (CA)

(21) Appl. No.: 13/478,664

(22) Filed: May 23, 2012

Related U.S. Application Data
(60) Provisional application No. 61/457,746, filed on May 25, 2011.

(51) Int. Cl. C07K 1/14; C07K 14/415

(52) U.S. Cl. 530/378

(57) ABSTRACT

A soy protein product having a protein content of at least about 60 wt % (N×6.25) d.b. is recovered in the processing of soy protein source material to form soy protein products wherein the soy protein source is extracted with calcium salt solution. The resulting soy protein solution is separated from the bulk of the residual soy protein source and then the soy protein solution is processed to remove finer residual solids, which are washed and dried to provide the soy protein product.

### REFERENCE TO RELATED APPLICATIONS

This application claims priority under 35 USC 119(e) from U.S. Provisional Patent Application No. 61/457,746 filed May 25, 2011.

### FIELD OF INVENTION

The present invention relates to the production of soy protein product, preferably soy protein concentrate.

### BACKGROUND TO THE INVENTION

In copending U.S. patent applications Ser. No. 12/603,087 filed Oct. 21, 2009 (US Patent Publication No. 2010-0098818 published Apr. 22, 2010 (S701)) and U.S. patent applications Ser. No. 12/923,897 filed Oct. 13, 2010 (US Patent Publication No. 2011-0038993 published Feb. 17, 2011 (S701CIP)), assigned to the assignee hereof and the disclosures of which are incorporated herein by reference, there is described the provision of a novel soy protein product having a protein content of at least about 60 wt % (N×6.25) on a dry weight basis, preferably a soy protein isolate having a protein content of at least about 90 wt % (N×6.25) d.b. The soy protein product has a unique combination of properties, namely:
completely soluble in aqueous media at acid pH values of less than about 4.4
heat stable in aqueous media at acid pH values of less than about 4.4
does not require stabilizers or other additives to maintain the protein product in solution
is low in phytic acid
requires no enzymes in the production thereof

In addition, the soy protein product has no beany flavour or off odours characteristic of soy protein products.

This novel soy protein product is prepared by a method which comprises:
(a) extracting a soy protein source with an aqueous calcium chloride solution to cause solubilization of soy protein from the protein source and to form an aqueous soy protein solution,
(b) separating the aqueous soy protein solution from residual soy protein source,
(c) optionally diluting the aqueous soy protein solution,
(d) adjusting the pH of the aqueous soy protein solution to a pH of about 1.5 to about 4.4, preferably about 2 to about 4, to produce an acidified clear soy protein solution,
(e) optionally concentrating the aqueous clear soy protein solution while maintaining the ionic strength substantially constant by using a selective membrane technique,
(f) optionally diafiltering the concentrated soy protein solution, and
(g) optionally drying the concentrated soy protein solution.

One of the key steps in producing the novel soy protein product discussed above is clarification of the aqueous soy protein solution formed in the extraction step. In general, a decanter centrifuge may be used to remove the bulk of the spent soy protein source from the aqueous soy protein solution. A disc stack centrifuge may be employed to remove finer solids not removed by the decanter centrifuge. In general, the solids recovered in the disc stack centrifuge may be combined with solids material discharged from the decanter centrifuge and the combined solids re-extracted to recover additional protein, dried and sold as animal feed, or simply discarded as waste.

### SUMMARY OF THE INVENTION

It has now been found that the finer solid material collected by the disc stack centrifuge may be washed and dried to provide a soy protein product having a protein content of at least about 60 wt % (N×6.25) d.b., preferably a soy protein concentrate, which may be used in a variety of applications.

Accordingly, in one aspect of the present invention, there is provided a method of forming a soy product, which comprises (a) extracting a soy protein source with an aqueous calcium salt solution to cause solubilization of soy protein from the protein source and to form an aqueous soy protein solution, (b) separating the aqueous soy protein solution from the bulk of the soy protein source, (c) applying a second separation step to the soy protein solution to recover finer residual solids not removed in the initial separation step, and (d) washing and drying the finer solids recovered in the second separation step to provide the soy protein product.

### GENERAL DESCRIPTION OF INVENTION

The initial step of the process of providing the soy protein isolate in the above-noted patent applications involves solubilizing soy protein from a soy protein source. The soy protein source may be soybeans or any soy product or by-product derived from the processing of soybeans, including but not limited to soy meal, soy flakes, soy grits and soy flour. The soy protein source may be used in the full fat form, partially defatted form or fully defatted form. Where the soy protein source contains an appreciable amount of fat, an oil-removal step generally is required during the process. The soy protein recovered from the soy protein source may be the protein naturally occurring in soybean or the proteinaceous material may be a protein modified by genetic manipulation but possessing characteristic hydrophobic and polar properties of the natural protein.

Protein solubilization from the soy protein source material is effected most conveniently using calcium chloride solution, although solutions of other calcium salts may be used. In addition, other alkaline earth metal compounds may be used, such as magnesium salts. Further, extraction of the soy protein from the soy protein source may be effected using calcium salt solution in combination with another salt solution, such as sodium chloride. Additionally, extraction of the soy protein from the soy protein source may be effected using water or other salt solution, such as sodium chloride, with calcium salt subsequently being added to the aqueous soy protein solution produced in the extraction step. Precipitate formed upon addition of the calcium salt is removed prior to subsequent processing.

As the concentration of the calcium salt solution increases, the degree of solubilization of protein from the soy protein source initially increases until a maximum value is achieved. Any subsequent increase in salt concentration does not increase the total protein solubilized. The concentration of calcium salt solution which causes maximum protein solubilization varies depending on the salt concerned. It is usually preferred to utilize a concentration value less than about 1.0 M, and more preferably a value of about 0.10 to about 0.15 M.

In a batch process, the salt solubilization of the protein is effected at a temperature of from about 1° C. to about 100° C., preferably about 15° C. to about 65° C., more preferably about 50° C. to about 60° C., preferably accompanied by agitation to decrease the solubilization time, which is usually about 1 to about 60 minutes, It is preferred to effect the solubilization to extract substantially as much protein from the soy protein source as is practicable, so as to provide an overall high product yield.

In a continuous process, the extraction of the soy protein from the soy protein source is carried out in any manner consistent with effecting a continuous extraction of soy protein from the soy protein source. In one embodiment, the soy protein source is continuously mixed with the calcium salt solution and the mixture is conveyed through a pipe or conduit having a length and at a flow rate for a residence time sufficient to effect the desired extraction in accordance with the parameters described herein, In such a continuous procedure, the salt solubilization step is effected, in a time of about 1 minute to about 60 minutes, preferably to effect solubilization to extract substantially as much protein from the soy protein source as is practicable. The solubilization in the continuous procedure is effected at temperatures between about 1° C. and about 100° C., preferably about 15° C. to about 65° C., more preferably between about 50° C. and about 60° C.

The extraction is generally conducted at a pH of about 4.5 to about 11, preferably about 5 to about 7. The pH of the extraction system (soy protein source and calcium salt solution) may be adjusted to any desired value within the range of about 5 to about 11 for use in the extraction step by the use of any convenient food grade acid, usually hydrochloric acid or phosphoric acid, or food grade alkali, usually sodium hydroxide, as required.

The concentration of soy protein source in the calcium salt solution during the solubilization step may vary widely. Typical concentration values are about 5 to about 15% w/v.

The protein extraction step with the aqueous salt solution has the additional effect of solubilizing fats which may be present in the soy protein source, which then results in the fats being present in the aqueous phase.

The protein solution resulting from the extraction step generally has a protein concentration of about 5 to about 50 g/L, preferably about 10 to about 50 g/L.

The aqueous calcium salt solution may contain an antioxidant. The antioxidant may be any convenient antioxidant, such as sodium sulfite or ascorbic acid. The quantity of antioxidant employed may vary from about 0.01 to about 1 wt % of the solution, preferably about 0.05 wt %. The antioxidant serves to inhibit oxidation of any phenolics in the protein solution.

The aqueous phase resulting from the extraction step then may be separated from the residual soy protein source, in any convenient manner, such as by employing a decanter centrifuge or any suitable sieve to remove the bulk of the residual soy protein source, followed by disc centrifugation and/or filtration, to remove the finer residual soy protein source material not removed in the initial separation step. The separated residual soy protein source may be dried for disposal. Alternatively, the separated residual soy protein source may be processed to recover some residual protein. The separated residual soy protein source may be re-extracted with fresh calcium salt solution and the protein solution yielded upon clarification combined with the initial protein solution for further processing as described below. Alternatively, the separated residual soy protein source may be processed by a conventional isoelectric precipitation procedure or any other convenient procedure to recover residual protein.

In accordance with the present invention, the residual protein source recovered in the second separation step of centrifugation and/or filtration may be washed with about 1 to about 20, preferably about 1 to about 5 volumes of water to remove residual extracted soy protein solution and contaminants and then dried by any convenient means to provide a soy protein product having a protein content of at least about 60 wt % (N×6.25) d.b., preferably a soy protein concentrate having a protein content of at least about 65 wt % (N×6.25) d.b,

The aqueous soy protein solution resulting from the clarification step may be subjected to the further processing steps described in the aforementioned U.S. patent applications Ser. Nos. 12/603,087 and 12/923,897 to form the novel soy protein product.

### EXAMPLE

This Example illustrates a procedure for producing a soy protein product from desludger solids.

30 kg of defatted soy white flake was added to 300 L of 0.13 M CaCl₂ solution at 60° C. and agitated for 30 minutes to provide an aqueous protein solution. The bulk of the residual soy white flake was removed with a decanter centrifuge and the resulting protein solution was further clarified with a disc stack centrifuge to produce 258 L of protein solution having a protein content of 2.69% by weight. 22.4 kg of watery solids was collected from the disc stack centrifuge. Residual liquid was removed from the watery solids by centrifugation on a lab scale centrifuge resulting in 6.8 kg of solids having a protein content of 14.98% by weight.

The solids were then suspended in 2 volumes of RO water and mixed thoroughly. This slurry was centrifuged at 6,000 g for 10 minutes to re-collect the solid material. The resulting liquid fraction containing residual salt and contaminants was discarded. This wash procedure was repeated a second time to remove more residual material and then the washed solids were freeze dried to yield 860 g of a protein product having a dry basis protein content of 77.98% (N×6.25).

### SUMMARY OF THE DISCLOSURE

In summary of this disclosure, a soy protein product, preferably a soy protein concentrate is produced as a by-product from the clarification of soy protein extract solution. Modifications are possible within the scope of the invention.

What we claim is:

1. A method of forming a soy protein product, which comprises:
(a) extracting a soy protein source with an aqueous calcium salt solution to cause solubilization of soy protein from the protein source and to form an aqueous soy protein solution,
(b) separating the aqueous soy protein solution from the bulk of the soy protein source,
(c) applying a second separation step to the soy protein solution to remove finer residual solids not removed by the initial separation step, and
(d) washing and drying the finer solids recovered in the second separation step to provide the soy protein product.

2. The method of claim 1 wherein the calcium salt is calcium chloride.

3. The method of claim I wherein the calcium salt solution has a concentration of less than about 1.0 M.

4. The method of claim 3 wherein the concentration of the calcium salt solution is about 0.10 to about 0.15 M.

5. The method of claim 1 wherein said extraction step is effected at a temperature of about 1° to about 100° C., preferably about 15° to about 65° C., more preferably about 50° to about 60° C.

6. The method of claim 1 wherein said extraction with aqueous calcium solution is conducted at a pH of about 4.5 to about 11, preferably about 5 to about 7.

7. The method of claim 1 wherein said aqueous soy protein solution has a protein concentration of about 5 to 50 g/L, preferably about 10 to about 50 g/L.

8. The method of claim 1 wherein said aqueous calcium salt solution contains an antioxidant.

9. The method of claim 1 wherein the solids recovered in the second separation step are washed with between about 1 and about 20, preferably about 1 to about 5 volumes of water.

10. The method of claim 1 wherein the finer solids recovered in the second separation step are washed and then dried to provide a soy protein product having a protein concentration of at least about 60 wt % (N×6.25) d.b.

11. The method of claim 10 wherein said solids are washed and dried to provide a soy protein concentrate having a protein concentration of at least about 65 wt % (N×6.25) d.b.
